(19) United States
(12) Patent Application Publication
(10) Pub. No.: US 2010/0331627 A1
(43) Pub. Date: Dec. 30, 2010

(54) ADHERENCE INDICATION TOOL FOR CHRONIC DISEASE MANAGEMENT AND METHOD THEREOF

(75) Inventors: Ajay Thukral, Indianapolis, IN (US); Stefan Weinert, Pendleton, IN (US); Robin Wagner, Indianapolis, IN (US); Maury Zivitz, Indianapolis, IN (US)

Correspondence Address: DINSMORE & SHOHL, LLP;FIFTH THIRD CENTER, ONE SOUTH MAIN STREET, SUITE 1300, DAYTON, OH, 45402, US

(73) Assignee: ROCHE DIAGNOSTICS OPERATIONS, INC., Indianapolis, IN (US)

(21) Appl. No.: 12/492,801

(22) Filed: Jun. 26, 2009

(51) Int. Cl. A61B 5/00

(52) U.S. Cl. 600/300

(57) ABSTRACT

An adherence indication tool for chronic disease self-management and method thereof for measuring adherence or compliance to following or achieving prescribed therapy steps to achieve stated target goals for improved chronic disease self-management are disclosed.

### FIELD OF THE INVENTION

Embodiments of the present invention relate generally to chronic disease management, and particularly to an adherence indication tool for chronic disease self-management and method thereof for measuring adherence or compliance to following or achieving prescribed therapy steps to achieve stated target goals for improved chronic disease self-management.

### BACKGROUND OF THE INVENTION

Achieving glycemic control to reduce long-term complications is the driving motivation of diabetic patients to monitor and self-manage the disease. The rules for such self-management are typically prescribed by a physician, such as an endocrinologist. It is expected that the rules for self-management be iteratively updated to maintain and improve glucose control. It is also expected that the patients are following the prescribed rules within reasonable limits. However, it has been observed from recent study results that diabetic patients have remarkably low adherence to prescribed therapy rules and low adherence to achievement of the therapy goal, thereby resulting in poor self-management of diabetes.

Some possible explanations for this low adherence are as follows: the prescribed therapy rules were a bad fit to a patient's disease state such that the patient adjusted insulin based on his or her own experience; the prescribed therapy rules were a good fit to the patient's disease state but instead the patient chose to follows his or her own rules; the prescribed therapy rules were a good fit to the patient's disease state but the patient was unable to follow them; the prescribed therapy rules were not a good fit to the patient's disease state yet the patient still followed the therapy rules with bad results; or the prescribed therapy rules were good a fit to the patient's disease state but the patient had a hard time in quantifying lifestyle data such as, for example, estimating a meal size. Other possible explanations may be that alternate states of the patient's disease, and/or the influence of other medications on the patient's disease state were not accounted for by explicit therapy rules.

### SUMMARY OF THE INVENTION

It is against the above background that embodiments of the present invention provide an adherence indication tool for chronic disease self-management and method thereof for measuring adherence or compliance to following or achieving prescribed therapy steps to achieve stated target goals for improved chronic disease self-management.

In one embodiment, disclosed is a method for measuring adherence to following or achieving prescribed therapy steps to achieve stated target goals for improved chronic disease self-management. The method comprises defining a plurality of adherence units, each adherence unit containing a plurality of rules governing activities which need to be accomplish in order to complete the prescribed therapy steps; collecting data when the activities are accomplished; specifying a time window of interest in the collected data; determining total number of adherence units in the collected data which fall within the specified time window of interest; counting each of the adherence units in the specified time window of interest as an adhered unit when the collected data indicates the accomplished activities were in accordance to the rules; determining adherence as a percentage of the count for the adhered units to the total number of adherence units for the specified time window; and providing at least one of the determined adherence percentage and adherence count for the specified time window.

In another embodiment an adherence indication tool measuring adherence to following or achieving prescribed therapy steps to achieve stated target goals for improved chronic disease self-management is disclosed. The adherence indication tool comprises a memory containing data collected when the activities were accomplished; a user interface facilitating selection of a plurality of adherence units, each adherence unit containing a plurality of rules governing activities which need to be accomplish in order to complete the prescribed therapy steps, and inputting of a specified time window of interest for the collected data; a process determining total number of adherence units in the collected data which fall within the specified time window of interest; a process counting each of the adherence units in the specified time window of interest as an adhered unit when the collected data indicates the accomplished activities were in accordance to the rules; a process determining adherence as a percentage of the count for the adhered units to the total number of adherence units for the specified time window; and an output providing at least one of the determined adherence percentage and adherence count for the specified time window.

These and other advantages and features of the invention disclosed herein, will be made more apparent from the description, drawings and claims that follow.

### BRIEF DESCRIPTION OF THE DRAWINGS

FIG. 1 depicts a therapy rule set based system shown with interacting subsystems;

FIG. 2 depicts a library of activities listed in a tabular format;

FIG. 3 depicts a library of protocols listed in a tabular format;

FIG. 4 depicts a list of activity codes of an individual on a timeline showing non-overlapping protocols;

FIG. 5 depicts a list of activity codes of an individual on a timeline showing protocol partially overlapped;

FIG. 6 depicts a list of activity codes of an individual on a timeline showing completely overlapping protocols;

FIG. 7 depicts an overall view of activity, data and time for a typical data scenario where colored circles represent the activity unit;

FIG. 8A depicts schematically activities only where the association to protocol group has been hidden;

FIG. 8B is a timeline depiction schematically showing a relation between adherence unit and activity units;

FIG. 9 is a timeline depiction showing an adherence unit covering multiple seasons;

FIG. 10 is a flowchart of a method according to an embodiment of the present invention;

FIG. 11 depicts a Diabetes Management System (DMS);

FIG. 12 depicts a graphical user interface provided on a display of a electronic device;

FIG. 13 depicts an example of output from an adherence indication tool showing a degree of adherence to therapy rules;

FIG. 14 is a example of graphical representation of adherence components reflecting the patients responsibility and physician responsibility in the overall concept of adherence; and

FIG. 15 is a graphical representation of adherence components reflecting the patients responsibility and physician responsibility in the overall concept of adherence.

### DETAILED DESCRIPTION

As will be appreciated by one of skill in the art, embodiments of the present invention may be provided as a method, a data processing system, or a computer program product. Accordingly, the present invention may take the form of an entirely hardware embodiment, an entirely software embodiment or an embodiment combining software and hardware aspects. Furthermore, the present invention may take the form of a computer program product on a computer-readable storage medium having computer-readable program code embodied in the medium. Any suitable computer medium may be utilized including hard disks, CD-ROMs, optical storage devices, magnetic storage devices, or programmable ROM devices.

It will be understood that each feature or combinations of features in the illustrations of the figures, can be implemented by computer-readable program code. The computer-readable program code may be loaded onto a general purpose computer, special purpose computer, or other programmable data processing device to produce a machine, such that the instructions contain in the code which execute on the computer or other programmable data processing apparatus create means for implementing the functions disclosed herein.

The computer-readable program code for implementing the present invention may be written in various object-oriented programming languages, such as Delphi and Java®. However, it is understood that other object oriented programming languages, such as NET, C++ and Smalltalk, as well as conventional programming languages, such as FORTRAN or COBOL, could be utilized.

There are a variety of solutions which facilitate better diabetes self-management. Such solutions are discussed in commonly owned and co-pending U.S. application Ser. Nos. 12/119,143; 12/119,201, and 12/491,523, all of which are incorporated fully herein by reference.

Central to the success of such solutions is the need for patient with diabetes (PwD) to be compliant and/or adhere to steps in a procedure, and more generally to follow therapy rule sets and meet algorithm requests. To further enhance such solutions, embodiments of the present invention may be used at a physician-patient level to evaluate which therapy rules hinder the achievement of therapy goals provided by such solutions.

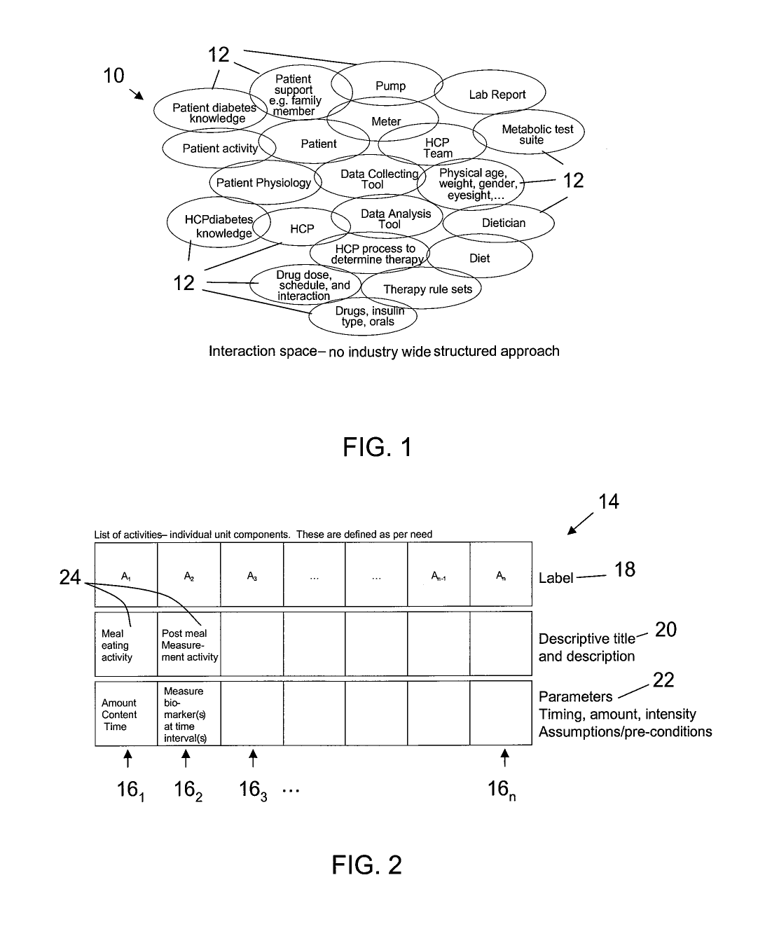

With reference to FIG. 1, a diabetes self-management system 10 is illustrated. As shown, the system 10 is normally is made up of interacting sub-systems 12, which it is to be appreciated has no industry wide structured approach to its organization or facilitation. Examples of such sub-systems 12 include, patient knowledge of their disease, patient support e.g. family helping the patient to self-manage the disease, the patient's activity or lifestyle, the patient's physiology, the patient physical attributes, the patient's health, the HCP knowledge of the disease, the process used by the HCP to determine therapy, drugs used, drug dose, drug dosing schedule, drug interactions of the patient, a patient's diet, experience of the dietician, patient test results, the medical team supporting the HCP and patient, the methods of collecting data, such as a bG meter, interactive devices, pumps, questionnaires, and the like, and the therapy rules resulting from a collecting of such sub-systems in order to met prescribed therapy goals. Accordingly, in one embodiment adherence rules discussed herein help in pinpointing which of such sub-systems 12 needs to be emphasized in order to improve/achieve prescribed therapy goals.

For example, in one specific embodiment, a HCP within the overall system 10 provides a therapy rule set by stepping through various sub-systems 12 to obtain information about the patient which he or she then analyzes or through further testing determines a suitable therapy for the patient that can help achieve therapy goals. The patient, in turn, then has a recommended method of performing an activity, such as diabetes self-management, within the overall system 10 via the prescribed therapy rule set and procedures. However, with disease self-management of a chronic disease such as diabetes, there are bound to be deviations from the recommended method (e.g. procedures, therapy rules). Accordingly, when the recommended method is altered to meet therapy goals there should be sufficient data to support such a decision.

It is to be appreciated that the recommended method provided for chronic disease self-management is made up of specified therapy steps (components). The specified therapy steps of the recommended method may be static or dynamic in nature. That is the specified therapy steps of the recommended method can be a function of time or other parameters such as, for example, if medication A is taken then medication B shall be substituted by medication C. An another example if medication A is taken then take medication B at time t1 minutes latter else if medication C is taken then take medication B at time t2 minutes latter. An another example of such a specified step is if a meal with higher fat composition is taken then the insulin dose shall be distributed with first insulin dose at time of meal intake shall be 80% of recommended insulin amount and the remaining insulin amount shall be injected 2 hours post prandial. However, it is to be appreciated that the specified therapy steps of the recommended method should state included rules (i.e., conditional statements, truth tables, etc.) explicitly such that the conformity to such rules can be evaluated.

In one embodiment, a medical adherence indication tool according to the present invention is provided that quantifies an aspect of adherence by allowing one to determine at a fundamental level for physician whether (1) the requirements for the recommended method were fulfilled, (2) which specified therapy step(s) of the recommended method did not satisfy the requirement, and (3) which aspect of the sub-system 12 has to be addressed. As adherence by default is patient centric, the adherence indication tool helps in determining which sub-systems 12 are road blocks in the recommended method to achieving a patient therapy goals. Measurement of adherence can also evaluate in other embodiments whether (1) the patient is adhering to the recommended method in general, whether (2) the therapy proposed by HCP is meeting the set therapy goals and consequently, whether (3) the therapy parameters and/or steps are appropriate. The measurement of adherence can also be used to identify potential sources of weakness in the management of a chronic disease, and to determine periods of time in a persons life when an additional support is needed. For example, during such a determined period, poor adherence is noted and then this poor adherence could be systematically addressed/corrected in some cases by additional 3^{rd} party help.

As discussed hereinafter, embodiments of the present provide a description of the degree of adherence of a patient to explicitly stated and prescribed therapy rules and a physician's targeted therapy goals for the patient. Other embodiments can help show the hidden relationships between such therapy rules and the targeted therapy goals for the patient as well. As also discussed hereinafter, in one embodiment a measurement of the degree of adherence by a person covers specified time windows. In particular, the degree of adherence shows to what extent certain step(s) or goal(s) were completed successfully within a selected time window of interest. In another embodiment, the measurement when applied to measure adherence for a chronically ill patient, such as a PwD, means measuring completion of a sequence of specified therapy steps that the patient is asked to follow in order to achieve or meet therapy goals set by a physician. By understanding the degree of adherence/compliance as facilitated by the embodiments of the present invention, a patient may achieve the following benefits: addresses the potential ambiguity between end users failure to comply versus methods effectiveness; provides end-user a quantified understanding of his/her action; and both a health care provider (HCP) and the patient have to do their part to achieve compliance as well as achieve therapy goals.

To help further explain the embodiments of the present invention, the following definitions of terms is provided. As used herein, the term “activity” means a unit of action consisting of explicitly defined steps which are a unique sequence/combination listed chronologically preferable. Each of the steps are a relevant part of the process/method to achieve the final outcome. An activity is understood to be explicitly stated by a physician, by a therapy application, such as activities which invoke events, by events which are defined on an at need per basis, or combinations thereof. Recording of activity related information provides valuable data on which analysis is carried out. As one suitable means for recording of events and for providing valuable data via analysis is disclosed by U.S. application Ser. No. 12/119,201, which is herein incorporated fully be reference, no further discussion is provided. By the above definition then the term “activity step” is understood to be a subpart of the activity.

With the above term definitions in mind, reference is now made to FIG. 2 which depicts a library 14 of activity units 16. Each column represents one of the activity units 16, and is described by an activity label 18, a descriptive title and description 20, and parameters 22. Each activity label 18 of the activity units 16 can be symbolically annotated by Aᵢ where i=1,2, and so on. The descriptive title and description 20 is used to describe the associated activity unit 16 by activity steps 24 that an individual e.g., the PwD or others associate with the PwD's therapy, has to perform. The activity steps 24 include a specific sequence, timeline and list of actions expected of the individual at each step. As an example, such activity steps 24 may include eating a meal, taking a post meal measurement, noting down information, entering information, measuring an activity step, doing the activity step, consuming specified drug of specified amount, consuming specified drug of specified amount at specified time, consuming specified drug of specified amount at specified time elapsed from specified occurrence of event and so forth. The parameters 22 enable quantifying each of the activity steps 24 to be performed and include aspects includes but not limited to timing, amount, duration and so forth.

Activity Timing

Even though an activity unit 16 is generally of finite duration, the start of activity 24 is considered as the absolute time for the activity unit 16. For example, a breakfast activity time is the time at which the breakfast activity unit is initiated. If the breakfast activity consists a number activity steps, such as for example, estimating carbohydrates in the breakfast meal, followed by measuring blood glucose (bG), followed by computation of insulin dose, followed by eating of the breakfast meal, followed by a 2-hour post-prandial measuring of bG, then the breakfast activity is timed as per preference or choice for marking the activity as preferably suggested by physician, so for example when the individual starts the estimation of the carbohydrate in the breakfast.

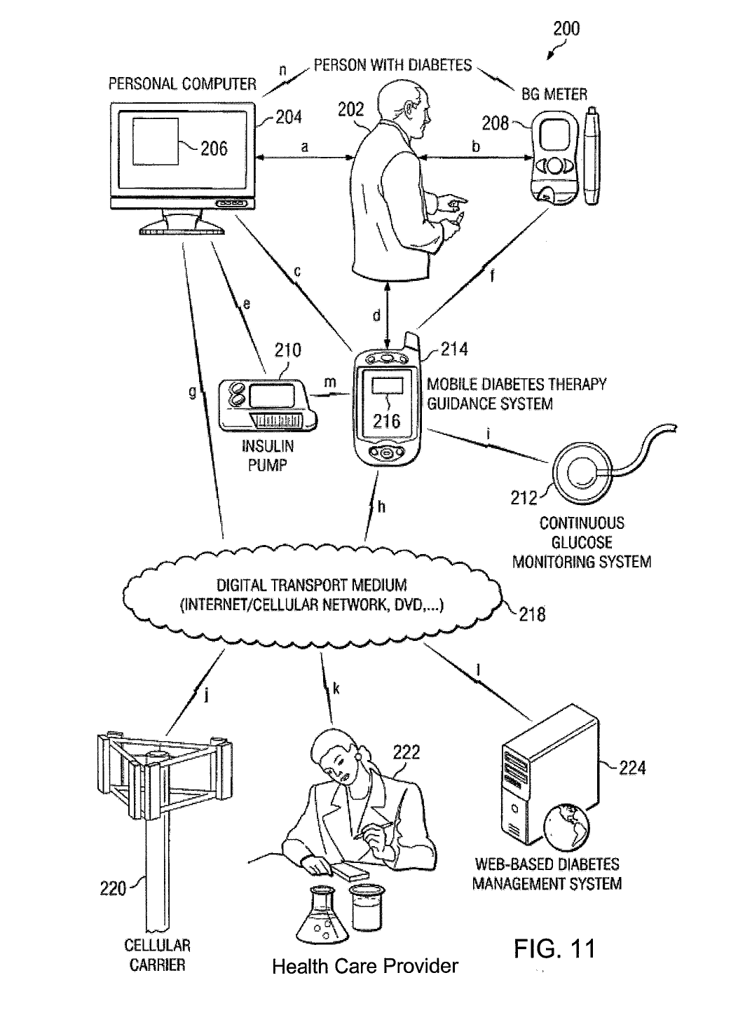

As used herein, an amount for a given activity step is an aspect of size or intensity or magnitude which describes it. Using the above breakfast example, the estimation of the size of carbohydrate is one measurement, and measuring bG concentration is another value which is recorded, for example via entering into a data collection unit (e.g., bG meter 204, FIG. 11). As used herein, duration of an activity step may implicitly have a start and end of the activity such as a meal one starts to eat and then completes when the individual stops ingesting meal. In this case such a case the activity duration may not be defined or is not generally relevant. However exercise may need an additional duration to specifically control the extent of exercise. Exercise is specified in intensity and needs duration where as meal is an amount with normally no time duration associated. An extreme case of activity duration is that no duration is specified when an activity continues for ever. From a practical viewpoint an infinitely continuing activity, such as breathing or a beating heart, may be of limited use but nevertheless covers a use-case. As used hereon, relative time of an activity step is defined as relative to either start of the activity or relative to another activity step, where the activity step is a sub-part of the activity.

Activity Example

A number of activity examples, and not limited thereto, are provided hereafter which are used to clarify the embodiments of the present invention. A meal amount activity is an activity example of when an individual weighs the meal and determines the carbohydrates in grams using a guideline book. As the guideline book is conventional no further discussion on it is provided. A meal insulin activity is an activity example of when the individual computes the meal insulin amount in units by multiplying the meal amount with an insulin to carbohydrate ratio factor supplied, for example, from the guideline book. For a pre-meal glucose measurement activity, the individual measures glucose within 10 minutes of meal intake via use of a glucose meter. It is to be appreciated that per typical measuring guidelines, a previous meal intake should be at least 4 hours earlier. For a post-meal glucose measurement activity, the individual measures glucose recommended about 2 hours after the start of the meal eating activity.

A meal eating activity is an activity example of when the individual eats a meal with balanced composition (as defined by a dietician) and which is at or larger than a specified minimum amount in one sitting at a regular pace. For example, the meal is completely ingested within 10 minutes, wherein non-glucose drinks can be sipped over a longer period of time, however glucose drinks should be restricted within the initial 10 minute time window. For an insulin dose activity, a computed insulin dose as per a dosing rule is injected. A physical activity is an activity example of when a physical activity leads to increased respiratory activity, increase in heart rate, or movement and exertion of limbs. Such a physical activity is normally quantified as a percentage increase from a previously established baseline to qualify as the physical activity (exercise). For fasting glucose, cessation of key external physiological excitation for a certain period of time occurs so that the measurement of glucose thereafter provides an accurate glucose concentration in resting state.

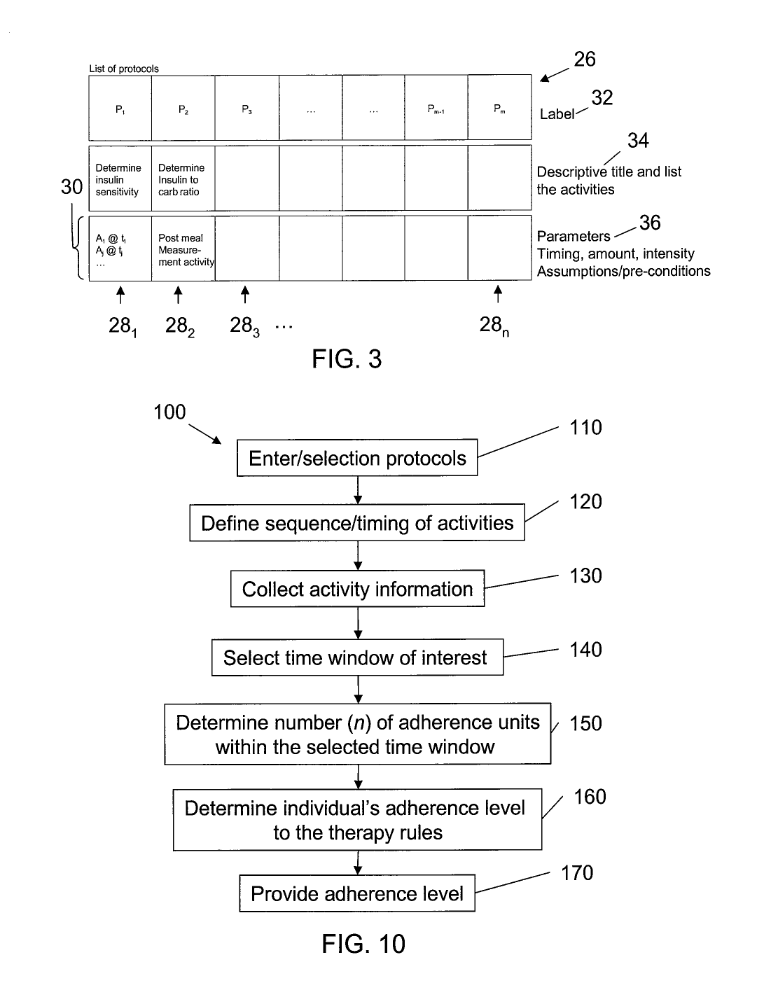

With the above activity examples in mind, reference is now made to FIG. 3 which depicts a library 26 of protocols 28. Each protocol 28 is a specific chronological set of protocol steps 30 each comprising activity unit 16 (FIG. 2) that an individual performs with specific medical outcome objectives in mind. Each column represents one of the protocols 28, and is described by a protocol label 32, a descriptive title 34, and a temporal specification 36 providing timing of the associated protocol steps 30 and the associated activity units 16 provided therewith. The protocol label 32 for each protocol 28 can be symbolically annotated by Aᵢ where i=1,2, and so on. The descriptive title 34 is used to describe collectively the protocol steps 30 associate with the protocol 28 to be performed. The temporal specification 36 include a specific timeline of the protocol steps 30, the sequence of the protocol steps 30, and what activity unit 16 at what time the individual is to perform for each of the protocol steps 30. For example as shown by FIG. 3, for protocol P₁—determine insulin sensitivity, the individual needs record information as prescribed by activity unit A₁ at time t₁, perform activity unit Aⱼ at time tⱼ, such as for example, consume a specified drug of a specified amount, and so forth.

Regarding absolute time of a protocol, it is to be appreciated that a protocol 28 is performed with the purpose of diagnosis, therapy determination, and/or prognosis. In most cases, activity selection and timing i.e., defining the temporal specification 36, is set up by the HCP (e.g., according to guidelines) for the specified protocol. Guidelines, for instance, may be derived from other published guidelines, experimental studies, data analysis of mathematical models or combinations thereof. The timing of the protocol can be an absolute time, may be independent of absolute time but may need to meet certain pre-conditions, or may be a combination of absolute time and pre-conditions. The protocol step 30 is defined as relative to either start of protocol 28 or relative to another protocol step.

As used herein, a “protocol performing requirement” describes an additional requirement in order to perform a protocol. For example, specifying the need for an additional person to assist the individual (such as if physically challenged) during the execution of the protocol is one example, of a protocol performing requirement. As also used herein, “protocol aborting requirements” are critical monitoring points specified in a protocol 28 which detail the aborting of the protocol if a situation occurs and which further specifies any additional recovery step as per the occurring situation. Examples of some protocols include, but not limited to, a determination of insulin sensitivity protocol which determines an individual's insulin sensitivity parameter for use in intensive therapy, and a determination of insulin to carbohydrate ratio protocol which determines an individual's insulin to carbohydrate ratio parameter for use also in the intensive therapy. Examples of how information for an individual is available for determination of adherence are now provided hereafter.

Timeline Schematics of Patient Activities and Protocols

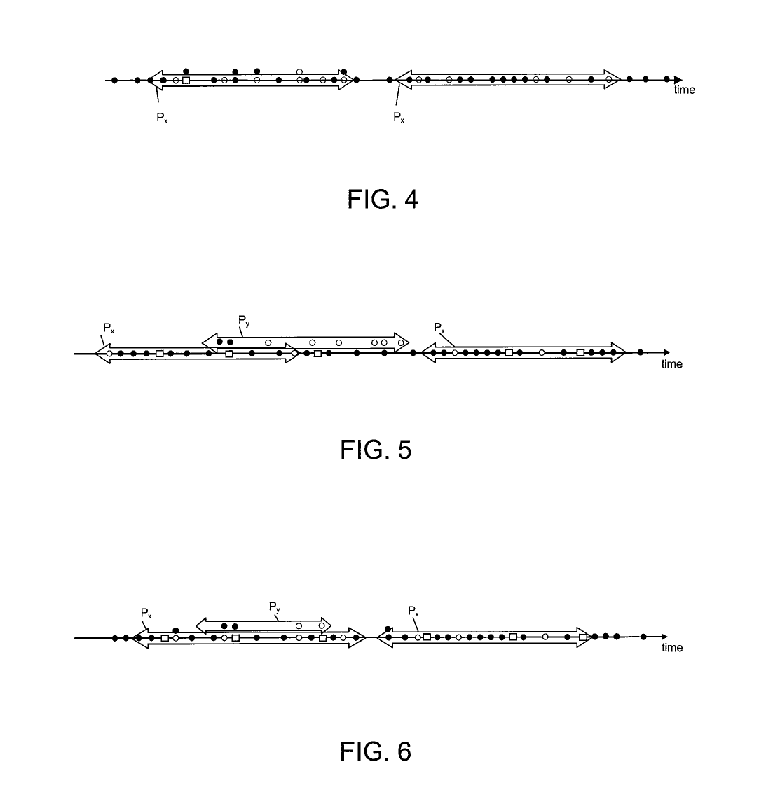

In a generic diabetes management system, an individual participates in enhancing their treatment by providing activity information (i.e., recording activities) and performing specific protocols. With references to FIGS. 4-6, such recorded activities are represented graphically by coded symbols e.g., white and black circles and squares with respect to one or more protocols. Each protocol is represented in FIGS. 4-6 by an arrowed segment i.e., a box segment with arrow heads at both the ends such as depicted. The various coded activities in relation to a protocol are defined as follows. If an activity is part of the protocol it is represented by a white circle. Such a white circle activity may happen to be a normal activity of the individual, but if the activity is a part of the protocol, then the activity is considered as required by the protocol and hence considered as part of the protocol. Black circles indicate normal activities of the subject which the subject is anticipated to continue to perform. These black circle activities do not impact the on going protocol activity, and as per the protocol are not mandatory or restricted by the protocol. If the black circle activity is part of the protocol then its association with the protocol is maintained.

Squares represent normal activities that are restricted by the protocol. That is the protocol explicitly states that certain activities cannot be performed while the protocol is in execution. In other words, a square activity is an activity that would have occurred or taken place if the associated protocol had not otherwise been running. Thus, the non-performance of the restricted activity is recorded and shown via the square in the timelines of FIGS. 4-6.

Each depicted timeline may cover a couple of days (or multiple days to weeks to months, if desired) with known activities identified by the above mentioned coded symbols (i.e., circles) and protocols by the arrowed segments. All the activities associated with the protocol are shown lying on or within the drawn boundaries of the protocol arrow (including any restricted activities). Overlapping activities are shown by stacking the coded symbols. It is to be appreciated that in other embodiment other forms of representing the above informs such via other coded symbols (squares, stars, numbers, colors, etc.), in tabular form, via bar graph, etc., may be used.

It is also to be appreciated that more than one protocol may potentially be executing simultaneously for an individual, wherein FIGS. 4-6 depicted different use cases of activities and protocols for an individual provided on a timeline. In FIG. 4, non-overlapping protocols Pₓ are shown. The start and end of each protocol Pₓ is shown on the timeline by the arrowed segment and the timing of each activity is shown by the placement of the coded circles on the timeline. These activities are referenced with respect to time as it has been discussed previously in an earlier section above. FIG. 4 also shows the various activities an individual may be performing during the time phase of each protocol Pₓ as well as when the protocols are not running. FIG. 4 also depicts the case when each protocol Pₓ is separated by some non zero time amount. The two protocols Pₓ may be sequenced in a specific order also such as, for example, the second protocol is done only if the first protocol has been performed successfully. Such arrangements and specific aspect are generally described as part of a pre-requisite to defining a protocol as decided by HCP with the individual.

In another use case of activities, FIG. 5 shows protocol Pₓ and P_{y} partially overlapped. Such a situation occurs when the second protocol P_{y} starts while the first protocol Pₓ has not completed. The two overlapping protocols Pₓ, P_{y} are such that for the duration of the time period of execution none of the activities specified within the arrowed segments impacts the outcome of the other protocol.

In still another use case of activities, FIG. 6 shows protocols Pₓ and P_{y} completely overlapped. Such a situation occurs when protocol Pₓ is started and the second protocol P_{y} is initiated and completed while the first protocol Pₓ is still running. As within the situation depicted by FIG. 5, the two overlapping protocols Pₓ, P_{y} are such that for the duration of the time period of execution none of the activities specified within each protocol (i.e., those activities laying on the arrowed segments) impact the outcome of the other protocol(s). Accordingly, FIGS. 5 and 6 depict situations where several activities may overlap. In general, activities are singular activities and the existence of two activities overlapping generally mean that in time these activities are in close proximity. However, as such activities can be completed in a short time duration of each other, for conveniences they are shown lumped together on the timeline. A discussion of the various embodiments of the present invention making used of such protocol and patient activity data is provided in later sections. A discussion on adherence is now provided hereafter.

Definition of Adherence

Adherence of rule(s) is defined in terms of a set of declarative sentences which when all are satisfied, results in an adhered unit (Λ) for the set of rule. Thus, if a rule is represented by ζᵢ where i=1, . . . n, then the truth set is ζ₁∩ζ₂∩ . . . ∩ζₙ. The collective set of rule(s) is defined as an adherence unit. Adherence is described as a percentage of adhered unit to the total number of adherence units (n) for a specified time window, and this is defined by equation (1) as follows:

$$Degree of Adherence = ( \frac{NumberOfAdheredUnit}{TotalNumberOfAdherenceUnit} ) 100 = \frac{{\sum }_{i = 1}^{n} {Λ}_{i}}{n} , \qquad ( 1 )$$

where Λᵢ is the i^{th} adherence unit where i=1, . . . n; Λᵢ computes to 1 if the adherence unit is adhered else it computes to 0. It is to be appreciated that the adherence unit Λᵢ is described as a collective unit of either one or more activity, one or more protocol step, or a combination of protocol step and activity. Within the given time window there are n such adherence units. In addition, it is to be appreciated that an adherence and compliance are used herein interchangeably; an activity is in general considered independent of its association to protocol; and an adherence unit test consists of evaluation of rules which in totem result in a value of either 0 or 1, where 1 represents an adhered unit. In short, rules describe how an adherence unit is evaluated. In this example the evaluation of Λᵢ is set to 0 or 1, but it is foreseen that this can be a real number.

Furthermore, a time period is the start and end of time describing the absolute time window during which all the recorded/documented activities are considered. Moreover, a subset time period is the subset of a time window within the time period. The subset time period covers events with certain periodicity. For example, a subset time period can be a breakfast activity covering Mondays only. In such an example, all meals and all snacks which are not a breakfast eaten on Mondays are excluded. Further time segmenting such as subset of subset and so forth are envisioned in other embodiments. Finally, a specified time window may consist one or more occurrences of adherence units to which adherence test is applied.

Lets consider a typical use case. A person in general working through the system 10 (FIG. 1) will be involved in managing his or her disease by facilitating the collection of contextual data for the various activities. The person will further be, from time to time, involved in performing structured testing (e.g., blood glucose testing) to determine a specific medical aspect. The person will therefore have data collected because of the protocol as well. As mentioned previously, an activity unit is associated with time. It is also associated with a protocol if the absolute time of the activity matches with the absolute time of the activity of the protocol, i.e. an activity can logically be associated in more than one place with respect to a protocol. If a time snapshot of the data is viewed then a typical data scenario is shown, for example, by FIG. 6.

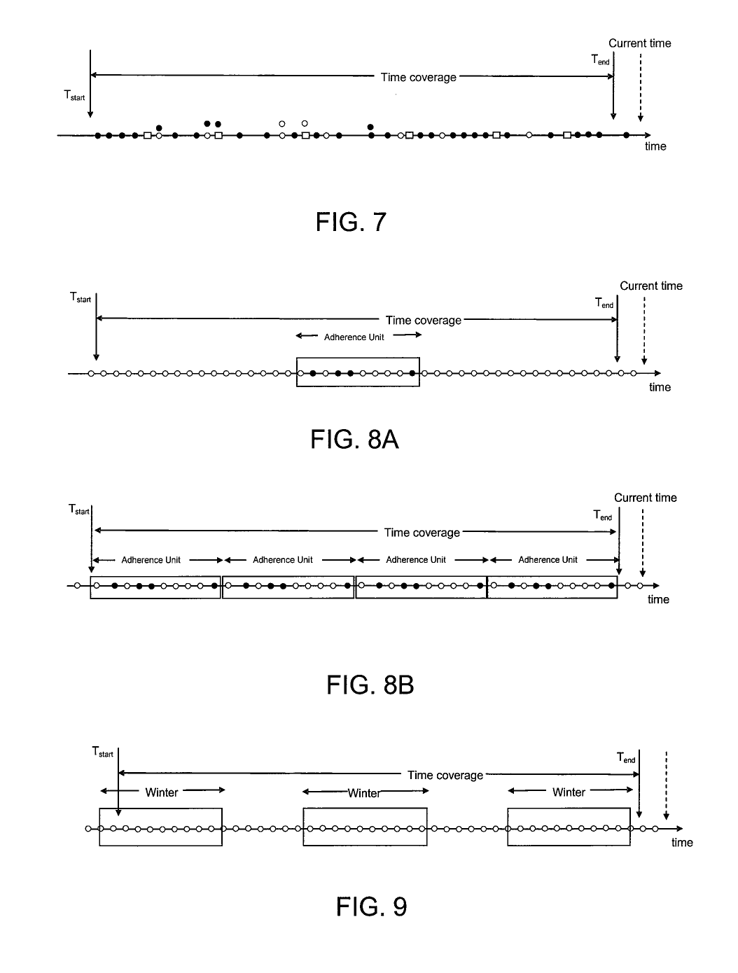

An activity in general can be examined independent of its association to groupings such as to protocols. From an adherence aspect, the activity unit is relevant. Accordingly, FIG. 6 is redrawn minus the protocol association as FIG. 7, where only activities are shown along the timeline, and the association to protocol(s) has been hidden. FIG. 8A shows an example of an adherence unit with associated activities where all the associations to the protocols have been hidden. In FIG. 8A several activity are selected by the box representing the adherence unit. With respect to time, the adherence unit and activities are not necessarily contiguous as some activities are not included in the selected adherence unit. It is to be appreciated that in FIG. 8A, the total number of adherence units (n) equals 1. FIG. 8B shows the adherence units that are covered in a selected time window of interest where then the total number of adherence units (n) equals 4. Another example is covered by FIG. 9, which shows a very large time window e.g., covering multiple seasons. In this example the circles represent an adherence unit, wherein the adherence unit is shown to cover multiple years.

To further explain what is considered an adherence unit Λᵢ, lets consider the example of performing intensive therapy for a meal. Approximately, the steps for performing intensive therapy for a meal are: (1) at meal time a non-zero amount of meal to be ingested is realized by the subject; (2) do a pre-meal bG measurement; (3) do carbohydrate count for the meal to be ingested, Amount; (4) compute meal-insulin amount, I_{M} given by I_{M}=I_{C}*Amount where I_{C} is insulin to carbohydrate ratio, Amount is grams of carbohydrates; (5) compute corrective-insulin amount, I_{CORR} given by

$${I}_{CORR} = \frac{bG - {bG}_{Target}}{{I}_{S}}$$

where I_{S} is insulin sensitivity, bG_{Target} is target bG at pre-meal time; (6) compute the total insulin dose, I_{M}+I_{CORR}; (7) deliver total insulin bolus 10 minutes prior to meal ingestion; and (8) ingest the stated meal 10 minutes subsequent to the insulin bolus. In the above example of performing intensive therapy for a meal, the adherence unit consist of steps 1 through 8. Alternatively, another adherence unit may be defined as just steps 3 and 4 listed in the above example. Other meaningful adherence units could further be derived/refined in order to better pertinent to the problem posed.

It is to be appreciated that adherence, unlike protocol, is an after the fact analysis which examines an aspect of individuals action as well as its targeted consequence (outcome). Adherence may simply look at the action or the outcome or both. Adherence may further consider more than one action, outcome or combination and provide an assessment of an individuals adherence level. As will be explained further in greater detail in a later section, adherence rules are then applied to a specified time window of the collected data.

Adherence Measuring

In one embodiment, a method for measuring adherence or compliance to following or achieving prescribed therapy steps to achieve stated target goals for improved chronic disease self-management, is generally indicated by symbol 100 in FIG. 10. In step 110, a computer program running on an appropriate processing device permits the HCP to input to or select from memory of the processing device protocols, and in step 120 to program the sequence and timing of associated activity units contained in each of the inputted or selected protocol(s) via a suitable user interface of the processing device. For example, the user interface can present for input or from memory the description detail of a protocol for selection, and after protocol entry or selection, then present for input or from memory the detailed description for each activity unit within the protocol for sequencing and timing. In step 130, the computer program when running on the processing device, instructs the processing device to collect data regarding an individual's activities per the prescribed (i.e., inputted and selected) protocol(s). The information regarding each activity is captured by the processing device in step 130 by the computer program instructing the processing device to prompt the individual via the user interface or other suitable output hardware and to accept user inputs providing the information. The computer program then stores the inputted information in memory of the processing device as collected data.

In one embodiment, the computer program annotates the collected data regarding the protocol and/or activity, such as with a timestamp of start and completion, contextual information, and other relevant quantified and subjective data. Recording of the activity and managing the associated information via the above mentioned data collection processes enables such data to be analysis in order to provide an assessment of an individuals adherence level, such as is discussed hereafter in later sections. In particular, via the data collection processes, the data information and associations are captured within the memory of the processing device (or a database) such that the recorded sequence of activities has no ambiguity. The collected data is then utilized in later steps for extracting relevant subset of data, applying adherence rules, and providing a number either as a ratio or in percentage format or an equivalent which indicates the extent to which adherence is accomplished.

After the above steps, a time window of interest is specified in step 140 to the computer program when a determination of adherence is desired. Normally the period of interest covers the time window from the current time and accounting for one or more previous days. The number of days can range from 1 days to multiple years. From a therapy perspective, a time window of 7 days to 90 days is normally considered; however, for the purpose of understanding disease progression or understanding other behavioral aspect, the time window may range in years. In addition, the time windows may be contiguous or non-contiguous. In particular, the time window is selected such that a question(s) such as, for example, “is patient compliant on Mondays”, “is patient compliant during weekends”, “is patient compliant during work days”, “is patient compliant during winter months” and so forth can be answered for each particular adherence unit, Λᵢ.

Next, in step 150, the computer program instructs the processing device to determine the number of adherence units Λᵢ in the collected data that fall within the specified time window, which represents the variable n in equation (1). For example, to further explain what is considered an adherence unit Λᵢ, lets consider the steps needed to perform intensive therapy for a meal. Approximately, the steps are: (1) at meal time a non-zero amount of meal to be ingested is realized by the subject; (2) do a pre-meal measurement; (3) do carbohydrate count for the meal to be ingested, Amount; (4) compute meal-insulin amount, I_{M} given by I_{M}=I_{C}*Amount where I_{C} is insulin to carbohydrate ratio, Amount is grams of carbohydrates; (5) compute corrective-insulin amount, I_{CORR} given by

$${I}_{CORR} = \frac{bG - {bG}_{Target}}{{I}_{S}}$$

where I_{S} is insulin sensitivity, bG_{Target} is target bG at pre-meal time; (6) compute the total insulin dose, I_{M}+I_{CORR}; (7) deliver total insulin bolus 10 minutes prior to meal ingestion; and (8) ingest the stated meal 10 minutes subsequent to the insulin bolus. In the above example of performing intensive therapy for a meal, the adherence unit consists of steps 1 through 8, wherein n=1. Thus, if the specified time window covered four such adherence units, for example, in a manner similarly shown by FIG. 8B, n then equals 4. Alternatively, another adherence unit may be defined as just steps 3 and 4 listed in the above example. Other meaningful adherence units could further be derived/refined in order to better pertinent to a problem posed.

Next in step 160, the individual's adherence level to the prescribed therapy rules is determined by the processing device via solving equation (1). The determined adherence level is then provided as output from the processing device in step 170, such as on the user interface or via other output hardware of the processing device. It is to be appreciated from equation (1) a degree of adherence can be determined for a number specific problems or questions. A few examples are provided hereafter.

Adherence to the Prescribed Therapy Rules

Table 1 provides an algorithm that describes a specific use case of method 100, where it is a desire to know the level of adherence of the individual in administering a prescribed amount of a drug during a specified time window. In performing the algorithm provided in Table 1, such as on the processing device, it is assumed that steps 110-150 of method 100 have been completed such that there are prescribed therapy rules, collected data, and a specified time window containing n number of adherence units. In addition, in Table 1, the term “ComputeDrugAmountAsPerRule” consists of therapy rules that the end user uses to compute the amount of drug he/she has to administer. In the current example the drug is insulin. The term “AdminsteredDrugAmount” is the actual amount of drug the end user administers to himself or herself. In the current example the drug is insulin. The term “DrugAmountTolerance” is a predetermined amount that the administers amount may differ from the computed amount of the drug. As shown, exceed this value will result in the conditional statement in Table 1 being false. This amount is set according to a prescribed therapy rule determined by, for example, by the HCP. The term “IsAdhered_Counter” is an integral counter of adhered occurrences(i.e., when the value for the DrugAmountTolerance amount is not exceeded). The term “NotAdhered_Counter” is an integral counter of occurrences where adherence is not met (i.e., when the value for the DrugAmountTolerance amount is exceeded). The term “DegreeOfAdherence” is the result of the algorithm, which in one embodiment is then provided as output from the processing device in step 170, such as on the graphical user interface or via other output hardware of the processing device. It is to be appreciated that other terms can be defined and substituted from the below mentioned in Table 1 such the a level of adherence of the individual in following or achieving prescribed therapy steps during a specified time window can be provided. As also provided in the example hereafter, a level of adherence in complying to a procedure likewise can be determined.

TABLE 1
For n number of adherence units:
If abs((ComputeDrugAmountAsPerRule) −
(AdministeredDrugAmount)) ≦ (DrugAmountTolerance)

IsAdhered_Counter = IsAdhered_Counter + 1

Else

NotAdhered_Counter = NotAdhered_Counter + 1;

End

$DegreeOfAdherence = \frac{IsAdhered\_Counter}{IsAdhered\_Counter + NotAdhered\_Counter}$

Compliance to a Procedure

A procedure is considered here as an adherence unit. As described earlier, the adherence unit consists of series of activities. These activities in general consists of collection of disparate activity such as, for example, measuring bG, measuring bG within a time window, dispensing drug, dispensing drug within time window, noting carbohydrate amount, noting fat amount, noting protein amount, eating meal within specified time window, and so forth. These activities as per definition of a protocol are pre-defined.

As in performing the algorithm provided in Table 1, the algorithm to determine the level compliance to a procedure listed in Table 2 also assumes that steps 110-150 of method 100 have been completed such that there are prescribed therapy rules, collected data, and a specified time window containing n number of adherence units. Using the “perform intensive therapy for a meal” procedure example used in a previous section above, an adherence unit for Table 2 is considered to include only step 8 i.e., ingest the stated meal 10 minutes subsequent to the insulin bolus. Accordingly, the terms used in the algorithm in Table 2 now provided a level of adherence of the individual to the rule of eating the meal or taking the insulin within the prescribed 10 minutes tolerance window, which is appropriately defined as the SetOfRule term in Table 2. As shown, if the SetOfRule term in Table 2 is satisfied, then the IsAdhered_Counter increments. Otherwise, the NotAdhere_Counter increments, and the algorithm repeats until every adherence unit in the specified time window is counted for compliance.

TABLE 2
For each of the adherence unit apply the rule(s)
If SetOfRules Satisfied

IsAdhered_Counter = IsAdhered_Counter + 1

Else

NotAdhered_Counter = NotAdhered_Counter + 1

End

$DegreeOfAdherence = \frac{IsAdhered\_Counter}{IsAdhered\_Counter + NotAdhered\_Counter}$

The term “DegreeOfAdherence” is the result of the algorithm in Table 2, which in one embodiment is then provided as output from the processing device in step 170, such as on the graphical user interface or via other output hardware of the processing device.

Another example of the SetofRule term may be taken from a procedure (method) to compute of an estimated HbA1C based on post-meal bG measurements. For such a method, each meal type (breakfast, lunch and supper) post meal bG measurements are needed. For best results, the specified time window is 60 days. Within that time window at least 45 measurements are needed for each meal, wherein the bG measurement for each meal occurs at 180 minute with a tolerance of plus/minus 30 minutes. The bG measurement around the time of measurement should be normally distributed. Accordingly, in such a procedure and in one embodiment, the SetofRule term could be defined to determined whether all the above measurement requirements/conditions are satisfied. In another embodiment, if the above rules are adhered then the estimated HbA1C can algorithmically be determined, such as for example, by the processing device if programmed with such a procedure. In another embodiment, the computed estimated HbA1C can be provided with the degree of adherence. One suitable estimated HbA1C procedure/method is disclosed by commonly owned and co-pending U.S. patent application Ser. No. 12/492,667, which is herein incorporated fully by reference.

Capturing Lifestyle

Similar to above example discussed in reference to Table 2, many of the above mentioned solutions which facilitate better diabetes self-management require the collection of lifestyle information. By lifestyle information it is information concerning an individual's habits and routines in self-managing his or her chronic disease. It is to be appreciated that such information may have many random variations. However, in order to better assess and recommend individualized solutions to improve therapy, a premise of statistical information is that the collected sample data set has to be representative of the population. Accordingly, rules are designed by such solutions in a manner that requests for information are dynamically triggered in order to assist the end user in effectively collecting such data concerning his or her lifestyle. In such solutions, an embodiment of the present invention is to define the SetofRules terms to determine whether the end user complied to triggered request for information in the specified time window and to give a degree of adherence for the specified time window.

Another example of such needed lifestyle information is to determine the meal eating habits of the patient by collecting such information. In some instances, the patient may be providing such information infrequently, and/or the timing and meal amount may be too random. In another embodiment of the invention, a number of SetofRules terms can be defined which determine whether the patient is providing such information at a recommend frequency, at recommend times, and which meet recommend amounts (within tolerances, if applicable).

In both the above cases, results are derived but the acceptance of the result is associated with a degree of adherence (°A). The idea is not to reject the result summarily but rather consider the information appropriately while making decisive conclusions. The information is still valuable. In context of lifestyle for e.g. an idea of lifestyle is still conveyed and some meaningful action can be done and over period of time the lifestyle information can be further strengthened. In still other embodiments, further generalization of each of the above cases can be made wherein the SetofRules term can be define on rules that use time and other parameters to update the therapy parameters, such that adherence or compliance to following or achieving such rules can be assessed. A discussion of a diabetes management system is now provided hereafter with reference made to FIG. 11.

Diabetes Management System (DMS)

In another embodiment, the method 100 as well as the above examples are facilitated as part of a Diabetes Management System (DMS), such as indicated generally by symbol 200 in FIG. 11. The DMS 200 helps an individual to self manage diabetes or provides diabetes care. In the area of diabetes care, there are multiple possible applications of the invention that provide value to the person with diabetes (PwD) 202 and to a health care provider (HCP) 222 that helps the PwD 202 with managing his or her diabetes. Typically, a person with diabetes (PwD) 202 and the HCP 222 will have multiple processing devices and software to help with the management of the PwD's disease. For example, it is assumed that the PwD 202 and/or the HCP 222 will have a personal computer 204 running a computer program 206 implementing method 100 as well as other software for tracking health status which includes blood glucose (bG) measurements and insulin dosing and so forth. The PwD 202 will also have a blood glucose (BG) meter 208 for intermittent bG measurements, and optionally an insulin pump 210 for delivering insulin subcutaneously, a continuous glucose monitoring system 212 for monitoring blood glucose frequently, which may be subcutaneous and/or cutaneous, and/or a mobile diabetes therapy guidance system 214 running therapy guidance software 216 which may or may not provide method 100, such as implemented on a mobile phone, a personal digital assistance, a notebook computer, and the like.

As shown by FIG. 11 and in one embodiment, the PwD 202 interacts directly (arrows a, b, d) with the personal computer 204, the bG meter 208, and optionally, the therapy guidance system 214. For the purpose of this example and in another embodiment, the insulin pump 210 and the continuous monitoring system 212 is also used by the PwD 202 and configured through the software 206 of the personal computer 204, the therapy guidance software 216 of the therapy guidance system 214 or the BG meter 208. It is to be appreciated that the processing device 204, 208, 210, 212, and 214 communicate (arrows e, c, f, and i) with each other in one form or another by a digital transport medium 218, such as via wired or wireless data communications, such wireless network 220, as well as with in another embodiment, a web-based Diabetes Management Systems 224, such as used by the HCP 222 and/or PwD 202. It is further to be appreciated that processing devices 204, 208, 210, 212, 214, and 224 can contain hardware and operating software/firmware that embodies the method 100 such as described previously above in earlier sections as well as in other embodiments clinical logic to facilitate the PwD 202 to self-manage his or her diabetes care therapy. With this DMS 200 in mind, a use case example is provided hereafter.

Use Case Example

It is to be appreciated that the PwD 202 must routinely meet with their HCP 222 for therapy assessment and updating. Typically, this patient-physician meeting occurs on a quarterly basis. A typical outcome of this meeting is an evaluation/determination of the following therapy parameters: (a) Basal rate setting on the pump is I_{Basal}(t) [U/h]; (b) Insulin to carbohydrate ratio is I_{C}[U/g]; (c) Insulin sensitivity is I_{S} [mg/dL/U]; (d) Glucose target G_{T} [mg/dL] which in general is function of time (or defined as per event); and (e) Pre meal glucose target range goals (G₁, G₂) [mg/dL] which in general is function of time (or defined as per event). Accordingly, on either the PC 204 or system 224, the HCP 222 can prescribes a number of therapy rules by programming them into any one of the processing devices of the diabetes management system (DMS) 200 which contains method 100.

For example, one of the therapy rules may be meal related therapy rules, which can be: (1) measure fasting bG; and (2) take pre-meal bG measurements. Rule (1) hopes to collect information regarding an early morning bG value and/or a pre-breakfast measurement. Accordingly, the computer program running of the processing device either triggers a reminder or the patient simply performs the bG measurement with the bG meter 208. Rule (2) covers the pre-meals which are the major meals of the day. In this example the patient has breakfast, lunch and supper as the major meals. For these events governed by Rule (2), the processing device is either programmed to trigger a reminder for bG measurement or alternatively the patient enters the events at the time of the initiation of the meal.

Other therapy rules may define a total meal related therapy, which is a well known intensive conventional therapy. For such a therapy, the meal rules are programmed as follows:
1) Identify/accept that a meal will be consumed. Either the event is reminded or the patient initiates the event.
2) Patient will next determine carbohydrate amount in the meal. The amount is entered in the processing device.
3) The processing device programmatically then recommends the meal related insulin. I_{M}=A_{CARB}I_{C} where I_{M} is the meal bolus insulin, A_{CARB} is the meal carbohydrate amount in gms and I_{C} is the Insulin required for covering each gram of carbohydrate. The patient will decide to accept or overwrite the recommended value.
4) Processing device then asks to either (i) Measure pre-meal glucose or (ii) use glucose value in the vicinity of the meal (while ensuring that the value satisfies the criteria for a data point at that instant).
5) Processing device calculates the total meal related insulin which is given by:

$${I}_{Total} = {I}_{M} + \frac{{I}_{S}}{( G - {G}_{T} )} .$$

The 2^{nd} term is the insulin adjustment to deviation between the target and pre-meal glucose values.
6) The patient either modifies or accepts the total meal related insulin recommendation.
7) Depending on the setup, the processing device will command an insulin pump to deliver the patient approved amount of insulin.

It is to be appreciated that above steps provide the insulin bolus needed to cover for the meal. This process, in general is expected to be followed for each meal event. In this embodiment, the adherence method 100 resides also on the processing device as a software implemented adherence indication tool, such as for example as an adherence module add-on to the running therapy guidance program or a stand-alone adherence indication tool application. The algorithm of the adherence method 100 in either implementation has access to the patient collected data such as the glucose values, meal information, time stamps, insulin data, therapy goal information, therapy rule information and so forth.

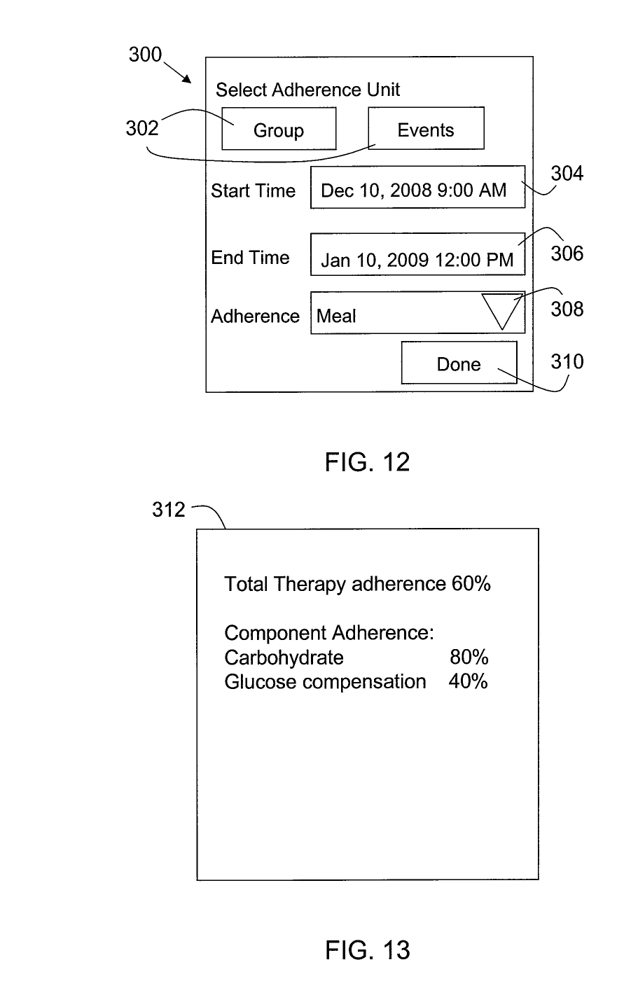

In one embodiment, the patient can review adherence at any time on the processing device or in another embodiment may be generated automatically as reliability indication to a provided value or parameter. When initiated, the adherence method 100 in one embodiment provides a graphical user interface 300 on a display of the processing device, such as depicted by FIG. 12. As shown the user can select a type for the adherence unit, e.g., group or events, via a selection input control 302. In addition the user may select start and end times for the specified time window via date-time input controls 304, 306, respectively. For example, in the illustrated embodiment, adherence in this example is measured in a specified time window starting at 9:00 am, Dec. 10, 2008 up to 12:00 pm of Jan. 10, 2009. Next, in the illustrated embodiment, the user selects to an activity on which is to see the level of adherence level using a drop down box 308 containing the available activities, and select a done button 310 in order for the method 100 to then determine the degree of adherence based on the inputted information and collected data.

For example, as discussed in this narrative, the embodiments of the present invention can enable the user to understand the following questions:
1) Against known patients lifestyle the patient inquires how many times he has adhered to monitoring meal activity;
2) Within the performed meal activity monitoring the adherence to therapy is evaluated;
3) Within the performed meal activity adherence to bG corrective therapy is evaluated;
4) Within the performed meal activity adherence to meal related insulin compensation is evaluated; and
5) Within the performed meal activity adherence the patient examines the adherence of the therapy to achieving the target goal. It examines statistical break down:
   a. When he/she adhered what is the ratio of achieved target
   b. When he/she did not adhere then what is the degree of therapy goal adherence
   c. Overall what is the degree of adherence goal

In the illustrated example of FIG. 12, the user has selected the meal adherence option. Accordingly, pre-defined to meal adherence the following aspects are examined by the method 100:
1) All major meals within the time window are identified. All major meals are the entries done by the patient for breakfast, lunch and supper.
2) For each meal the therapy rule is examined. The therapy rule is made up of compensation for carbohydrate and compensation for glucose. The total meal related insulin is the sum of the two.
3) Adherence examines the difference between the user specified insulin amount for the meal to the amount that the therapy rules recommend. The difference is reported as a percentage

$$DegreeofAdherence = ( \frac{NumberofAdhereOccurence}{TotalNumberofOccurence} ) 100 \%$$

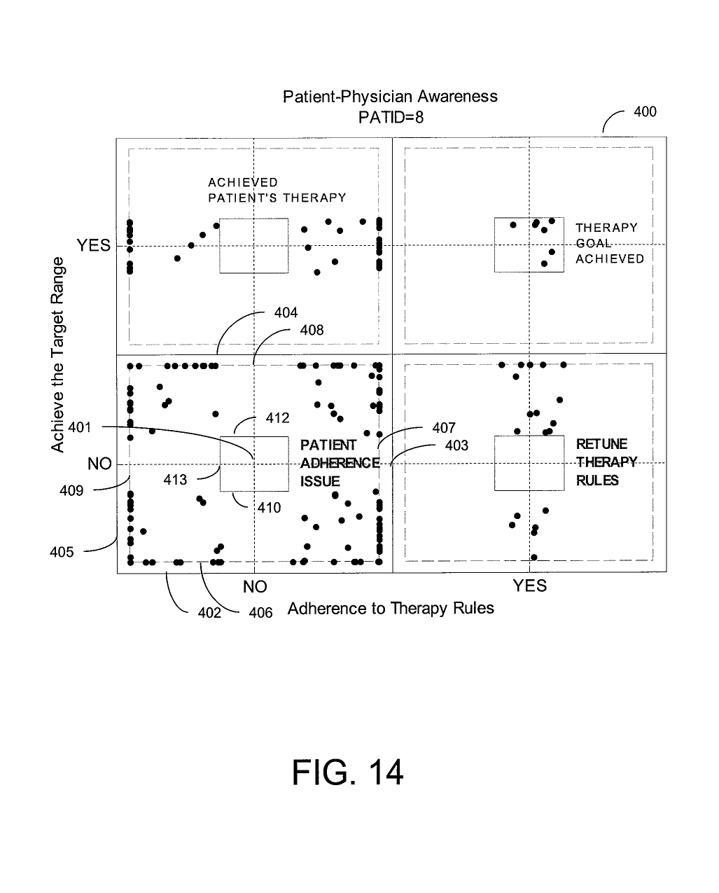

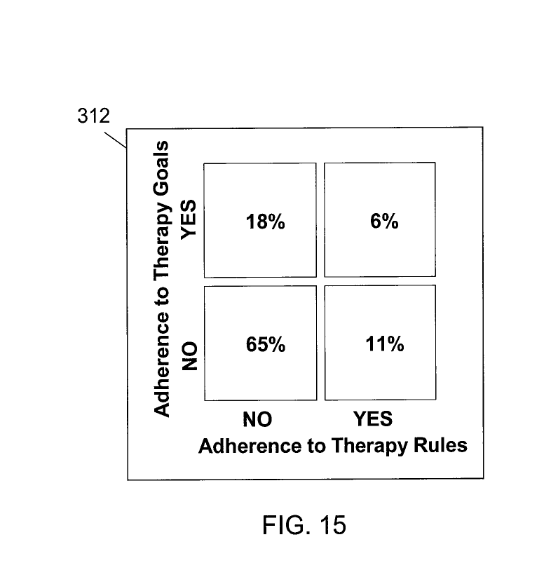

As an example, output 312 from adherence indication tool is shown by FIG. 13, which can be provided on the display of the processing device. As shown, the Total therapy adherence is 60%, and the components adherence such as to carbohydrate and glucose compensation is 80% and 40%, respectively. Additional output providing a graphical representation of adherence to therapy rule(s) and therapy goal(s) are further shown by FIGS. 14 and 15.

FIG. 14 is output of the method 100 in which adherence components reflecting the patients responsibility and physician responsibility in the overall concept of adherence is graphically represented. FIG. 14 also visualizes how far off values in the collected data are from desired ranges. From a categorical aspect of decision making, FIG. 14 shows a number of instances associated with each Yes, No combinations of whether target goal(s) have been achieved and therapy rules have been adhered, as well as the intensity/quantity of adherence. The second information overlaid uses the continuous information and shows the degree of deviation.

For example, consider the No-No grid of FIG. 14 where the vertical (y-) axis is the achieving the therapy goal and the horizontal (x-) axis is adherence to therapy rules (the section of plot enclosed by lines marked 402, 403, 404, 405). In this example all points that fail to satisfy both therapy target range for pre-prandial is (G₁, G₂) and correct insulin amount I₀ with acceptable tolerance of 0.5 are plotted. All the data points lying in this section of plot are shifted and scaled before the points are plotted. The center of the section of the No-No plot is (1,1) and the sides of the section of No-No are at ±1 with respect to the (1,1). The other sections are similarly created by translating the data and then scaling the data. So Yes-No is at (3,1) with outer box at ±1, No-Yes is at (1,3) with outer box at ±1 and Yes-Yes is at (3,3) with outer box at ±1. Further going back to the example (the No-No grid), then the following attributes of the graph are:
1) Set

$$\frac{{G}_{1} + {G}_{2}}{2}$$

as the origin (401). The origin lets label it as G₀;
2) Define vertical scale factor

$$K = \frac{1}{4} ( \frac{1}{abs ( {G}_{0} - {G}_{1} )} ) .$$

Note, the scale factor normalizes the contributions from the different data points to enable a comparison within a particular “Yes/No” quadrant;
3) Define solid horizontal lines of inner box with respect to G₀ as

$$K ( {G}_{1} - {G}_{0} ) = - \frac{1}{4}$$

(segment 410) and

$$K ( {G}_{2} - {G}_{0} ) = \frac{1}{4}$$

(segment 412);
4) The solid horizontal lines for outermost box are drawn at ±1 (segments 402 and 404);
5) The horizontal dashed lines of the inner box are drawn at ±0.9 (segments 406 and 408); and
6) The black dots for (No, No) part are then bG values for which neither therapy rules nor the therapy target were satisfied within the time window of interest. Thus the black dots are bG values satisfying the selection criteria and computing the value:

$${y}_{i} = \{ \begin{matrix}{y}_{NO , NO}^{0} - 0.9 & if K ( {G}_{1} - {G}_{i} ) < - 0.9 \\ {y}_{NO , NO}^{0} + K ( {G}_{1} - {G}_{i} ) & if - 0.9 \geq K ( {G}_{1} - {G}_{i} ) \leq 0.9 \\ {y}_{NO , NO}^{0} + 0.9 & if K ( {G}_{1} - {G}_{i} ) > 0.9\end{matrix} \\ Where \\ {y}_{NO , NO}^{0} = 1.$$

A similar computation was conducted for therapy rules. In this it is the insulin amount. The insulin amount itself can be variable depending on the meal size and bG value. However as per therapy rule there is a correct insulin amount I₀ which is the origin (marked 401). The allowed insulin amount error, in our example is set to ±0.5 which is (I₁=I₀−0.5, I₂=I₀+0.5). Then as described for therapy goals we have similar computation for therapy rule:
1) Set

$$\frac{{I}_{1} + {I}_{2}}{2}$$

as the origin (401). The origin lets label it as I₀;
2) Define horizontal scale factor

$$K = \frac{1}{4} ( \frac{1}{{I}_{0} - {I}_{1}} ) ;$$

3) The solid vertical lines of inner box are with respect to G₀ as

$$K ( {I}_{1} - {I}_{0} ) = - \frac{1}{4}$$

(segment 413) and

$$K ( {I}_{2} - {I}_{0} ) = \frac{1}{4}$$

(segment 411);
4) The solid vertical lines of outermost box are drawn at ±1 (segments 403 and 405);
5) The dashed vertical lines of inner box are drawn at ±0.9 (segments 407 and 409);
6) The black dots for (No, No) part are then insulin amount for which neither therapy rules nor the therapy target were satisfied within the time window of interest. Thus the black dots are insulin amount satisfying the selection criteria and computing the value:

$${x}_{i} = \{ \begin{matrix}{x}_{NO , NO}^{0} - 0.9 & if K ( {I}_{1} - {I}_{i} ) < - 0.9 \\ {x}_{NO , NO}^{0} + K ( {I}_{1} - {I}_{i} ) & if - 0.9 \geq K ( {I}_{1} - {I}_{i} ) \leq 0.9 \\ {x}_{NO , NO}^{0} + 0.9 & if K ( {I}_{1} - {I}_{i} ) > 0.9\end{matrix} \\ Where \\ {x}_{NO , NO}^{0} = 1.$$

To actually draw the points for the No-No grid then we have (xᵢ+1, yᵢ+1), where (xᵢ, yᵢ) are scaled coordinates with respect to (G₀, I₀). Similarly other grid points are obtained and then plotted as follows:
1) for No-No [symbol] (xᵢ+1, yᵢ+1)
2) for No-Yes [symbol] (xᵢ+1, yᵢ+3)
3) for Yes-No [symbol] (xᵢ+3, yᵢ+1)
4) for Yes-Yes [symbol] (xᵢ+3, yᵢ+3)

Accordingly, by the example illustrated by FIG. 14, a patient having their data points falling into the No-No box will have to comply to the prescribed therapy rules in order to realize any progress. A patient having data points falling into the Yes-No box is achieving the desired target range but the therapy rules do no reflect the patient's approach to achieving the desired target range. Such a situation may indicate that the patient understands how to self-manage their disease states. Accordingly, the HCP should interview the patient such that the patient's methodology used to achieve the desired therapy goal and/or interpretation of the prescribed therapy rules can be documented and better understood. A patient having data points falling into the No-Yes box is following the rules but the therapy rules will have to be modified by the HCP as such rules do not permit the patient to achieve the desired target range. A patient having data points falling into the Yes-Yes box is following the rules and achieving the desired target range, and thus no modification is needed.

Alternatively, the information graphically represented by FIG. 14, can represented by numerical values as shown in FIG. 15. FIG. 15 shows that the plot is a composite representation of two kinds of information. The first one is the categorical combination of the result: Yes, No with respect to the two conditions of adherence to therapy rules and adherence to therapy goals. This results in a 2 by 2 grid shown by FIG. 15, whereby the maximum degree of compliance is 100%.

In still another embodiment, an extension of the method 100 is wherein the therapy rules also are a function of time. In such an embodiment, for example, in certain therapy solutions the user can modify the therapy settings based on monitored data. For example, the therapy rule consists of changing basal insulin as per the following rule: increment the current basal insulin value by 10% if the fasting value over the last 7 days is greater than the target fasting values. The determination of the degree of adherence then follows the same principle of computing the adherence which now become functions of time. Furthermore, with appropriate math adjustment to adherence equation (1), the graphical representation presented in FIG. 14 can still be used when normalized to the values. There are other variations such as, for example, off center and so forth in which to explain other various plotting combinations.

Having described the disclosure in detail and by reference to specific embodiments thereof, it will be apparent that modifications and variation. For example, although the systems and methods disclosed herein for chronic disease self-management has been described primarily with respect to diabetes, the invention may also be applied to other chronic disorders and diseases, such as such as heart/cardiovascular diseases, cancer, and chronic respiratory diseases without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

What is claimed is:

1. A method for measuring adherence to following or achieving prescribed therapy steps to achieve stated target goals for improved chronic disease self-management comprising:
defining a plurality of adherence units, each adherence unit containing a plurality of rules governing activities which need to be accomplish in order to complete the prescribed therapy steps;
collecting data when the activities are accomplished;
specifying a time window of interest in the collected data;
determining total number of adherence units in the collected data which fall within the specified time window of interest;
counting each of the adherence units in the specified time window of interest as an adhered unit when the collected data indicates the accomplished activities were in accordance to the rules;
determining adherence as a percentage of the count for the adhered units to the total number of adherence units for the specified time window; and
providing at least one of the determined adherence percentage and adherence count for the specified time window.

2. The method of claim 1 further comprises selecting from a library contained in memory of a processing device the plurality of rules governing activities.

3. The method of claim 1 further comprises inputting into memory of a processing device the collected data.

4. The method of claim 1 further comprises setting a sequence and timing of the activities contained in each of the adherence units.

5. The method of claim 4 further comprising providing a user interface of a processing device by which to set the sequence and timing of the activities in each of the adherence units.

6. The method of claim 1 further comprises using a processing device to perform the collecting of the data.

7. The method of claim 1 further comprises programming a processing device to prompt for information regarding each of the activities when accomplished.

8. The method of claim 1 further comprises storing the collected data in memory of a processing device.

9. The method of claim 1 further comprises annotating the collected data with a timestamp of start and completion.

10. The method of claim 1 wherein the specified time window of interest is defined by a starting time and date, and an ending time and date.

11. An adherence indication tool measuring adherence to following or achieving prescribed therapy steps to achieve stated target goals for improved chronic disease self-management comprising:
a memory containing data collected when the activities were accomplished;
a user interface facilitating selection of a plurality of adherence units, each adherence unit containing a plurality of rules governing activities which need to be accomplish in order to complete the prescribed therapy steps, and inputting of a specified time window of interest for the collected data;
a process determining total number of adherence units in the collected data which fall within the specified time window of interest;
a process counting each of the adherence units in the specified time window of interest as an adhered unit when the collected data indicates the accomplished activities were in accordance to the rules;
a process determining adherence as a percentage of the count for the adhered units to the total number of adherence units for the specified time window; and
an output at least one of the determined adherence percentage and adherence count for the specified time window.

12. The adherence indication tool of claim 11 further comprises a library of the plurality of rules governing the activities contained in the memory.

13. The adherence indication tool of claim 11 further comprises an input for receiving into the memory the collected data.

14. The adherence indication tool of claim 11 wherein the user interface is used to set a sequence and timing of the activities contained in each of the adherence units.

15. The adherence indication tool of claim 11 wherein the output providing the determined adherence percentage is a display.

16. The adherence indication tool of claim 11 further comprises an external processing device which performs the collecting of the data and provides the collected data to the adherence indication tool.

17. The adherence indication tool of claim 16 wherein the collected data is provided over a network and where said adherence tool is implemented on a diabetes management system.

18. The adherence indication tool of claim 11 further comprises being implementing on a portable processing device.

19. The adherence indication tool of claim 11 further comprises being implementing on a portable processing device which collects the data.

20. The adherence indication tool of claim 11 wherein user interface enables the input of a starting time and date, and an ending time and date for the specified time window of interest.
